(19) United States
(12) Patent Application Publication
(10) Pub. No.: US 2014/0194829 A1
(43) Pub. Date: Jul. 10, 2014

(54) DOSE MEASURING DEVICE FOR DRUG DELIVERY DEVICE

(71) Applicant: Samsung Electronics Co., Ltd., Suwon-si (KR)

(72) Inventors: In-Geol BAEK, Icheon-si (KR); Jae-Kyung KWAK, Ansan-si (KR); Sang-Hun LEE, Yongin-si (KR)

(73) Assignee: Samsung Electronics Co., Ltd., Suwon-si (KR)

(21) Appl. No.: 14/151,204

(22) Filed: Jan. 9, 2014

(30) Foreign Application Priority Data
Jan. 10, 2013 (KR) 10-2013-0002760

(51) Int. Cl. A61M 5/315; A61B 19/00

(52) U.S. Cl. CPC A61M 5/31568; A61M 5/31556; A61B 19/46
USPC 604/207; 73/861

(57) ABSTRACT

A dose measuring device for a drug delivery device is provided. The dose measuring device includes a main body and a dial sleeve coupled to the main body to spirally move, the dose measuring device including a housing provided to be removable from the main body, a rotation ring received in the housing to rotate, an encoder installed in the housing to detect the amount of rotation of the rotation ring, and an auxiliary sleeve provided to be removable from the dial sleeve and to spirally move together with the dial sleeve in the housing, in which the rotation ring is bound to the auxiliary sleeve to rotate together with the auxiliary sleeve and is fixed in the housing in a longitudinal direction of the main body.

### CROSS-REFERENCE TO RELATED APPLICATION(S)

This application claims the benefit under 35 U.S.C. §119(a) of a Korean patent application filed on Jan. 10, 2013 in the Korean Intellectual Property Office and assigned Ser. No. 10-2013-0002760, the entire disclosure of which is hereby incorporated by reference.

### TECHNICAL FIELD

The present disclosure relates to a drug delivery device. More particularly, the present disclosure relates to a device for measuring a dose of a drug delivered through a drug delivery device.

### BACKGROUND

Generally, diabetic patients have problems with insulin secretion in their bodies, and thus they need to continuously measure blood pressure and even inject insulin if necessary. Recently, a portable drug delivery device has been provided to allow the users to inject insulin for themselves. An insulin pump or insulin pen may be an example of the drug delivery device. A structure of the insulin pen is disclosed in Korean Patent Registration No. 1,124,194 (registered on Feb. 29, 2012, and corresponding to International Patent Publication No. WO 2004/082748 published on Sep. 30, 2004). In the insulin pen, drug doses are usually indicated on a dial sleeve, such that the users may decide a drug dose for themselves.

An inappropriate insulin dose may aggravate the user's symptoms or disease, and thus it is desirable to record details regarding a dose, injection date and time, and so forth. The user's doctor may determine the condition of a disease of the user by referring to information such as the recorded dose, injection date and time, and so forth. However, current drug delivery devices, such as the insulin pen, is structured merely to allow users to determine doses for themselves, and the users have to directly record information such as injection date and time, a dose, an accumulated dose, and the like. If the user fails to record some information, such as a dose, the user may incorrectly determine a dose at future injection, worsening the user's symptoms or disease. Therefore, it is urgent to provide a drug delivery device that allows measurement and recording of a dose without user's direct recording. However, with drug delivery devices that have already been commercialized and sold to users or have already been released in the market, the users may adjust a dose, but measurement and recording of a dose are substantially impossible, such that the users have to directly record information such as a dose.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide a dose measuring device that is removable from a commercialized drug delivery device without physical modifications or design changes with respect to the drug delivery device.

In accordance with an aspect of the present disclosure, a dose measuring device for a drug delivery device that includes a main body and a dial sleeve coupled to the main body to spirally move. The dose measuring device includes a housing arranged to receive the main body of the drug delivery device, a rotation ring arranged in the housing to rotate, an encoder installed in the housing to detect an amount of rotation of the rotation ring, and an auxiliary sleeve arranged to be removable from the dial sleeve and to spirally move together with the dial sleeve of the drug delivery device in the housing, in which the rotation ring is bound to the auxiliary sleeve to rotate together with the auxiliary sleeve and is fixed in the housing in a longitudinal direction of the main body.

In accordance with another aspect of the present invention, a drug delivery device is provided. The drug delivery device includes a main body, a dial sleeve coupled to the main body so as to rotate and including an adjustment grip on one end portion, and an injection button pressable by the user to operate the drug delivery device to deliver a drug to the user's body, wherein binding protrusions are formed on an outer circumferential surface of the dial sleeve so as to engage with an inner surface of an auxiliary sleeve of a dose measuring device, such that the auxiliary sleeve of the dose measuring device spirally moves together with the dial sleeve.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a perspective view illustrating a dose measuring device for a drug delivery device according to an embodiment of the present disclosure;

FIG. 2 is a side view illustrating a dose measuring device according to an embodiment of the present disclosure;

FIG. 3 is a side view illustrating a housing of a dose measuring device according to an embodiment of the present disclosure;

FIG. 4 is a side view illustrating a state in which a housing is removed from a dose measuring device according to an embodiment of the present disclosure;

FIG. 5 is a perspective view illustrating a rotation ring of a dose measuring device according to an embodiment of the present disclosure;

FIGS. 6 and 7 are diagrams for describing an encoder of a dose measuring device according to an embodiment of the present disclosure;

FIG. 8 is a perspective view illustrating an auxiliary sleeve of a dose measuring device according to an embodiment of the present disclosure;

FIGS. 9 and 10 are perspective views illustrating a guide member of a dose measuring device according to an embodiment of the present disclosure;

FIGS. 11 and 12 are perspective views illustrating a fixing member of a dose measuring device according to an embodiment of the present disclosure;

FIG. 13 illustrates a drug delivery device configured to be removable from a dose measuring device according to an embodiment of the present disclosure;

FIG. 14 is a side view illustrating a state in which a dose measuring device is mounted on a drug delivery device according to an embodiment of the present disclosure; and

FIG. 15 is a side view illustrating a state in which a housing is removed from a dose measuring device according to an embodiment of the present disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the present disclosure. Accordingly, it should be apparent to those skilled in the art that the following description of various embodiments of the present disclosure is provided for illustration purposes only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

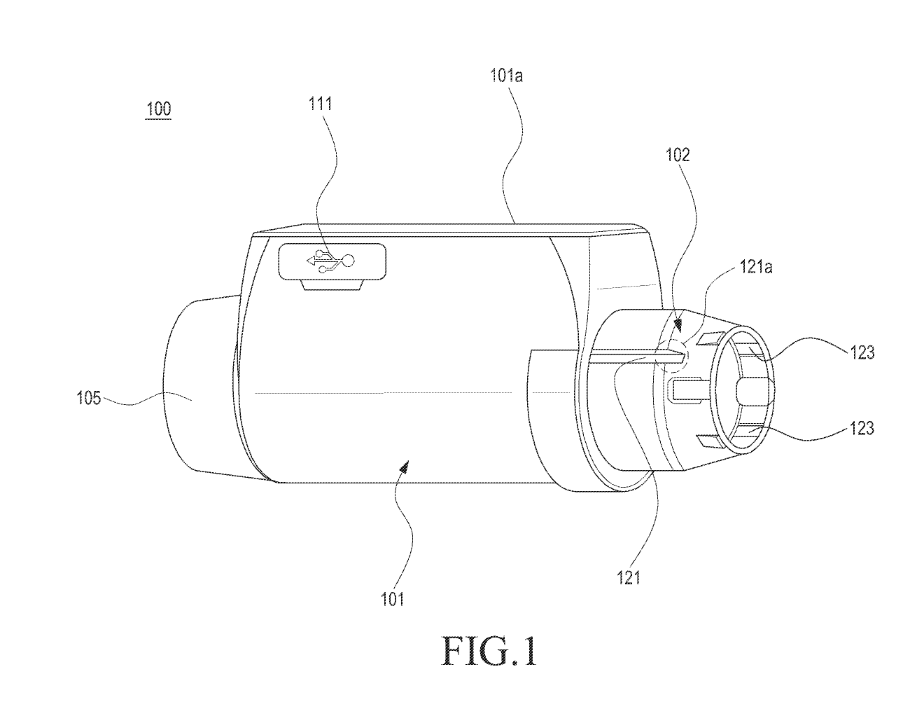

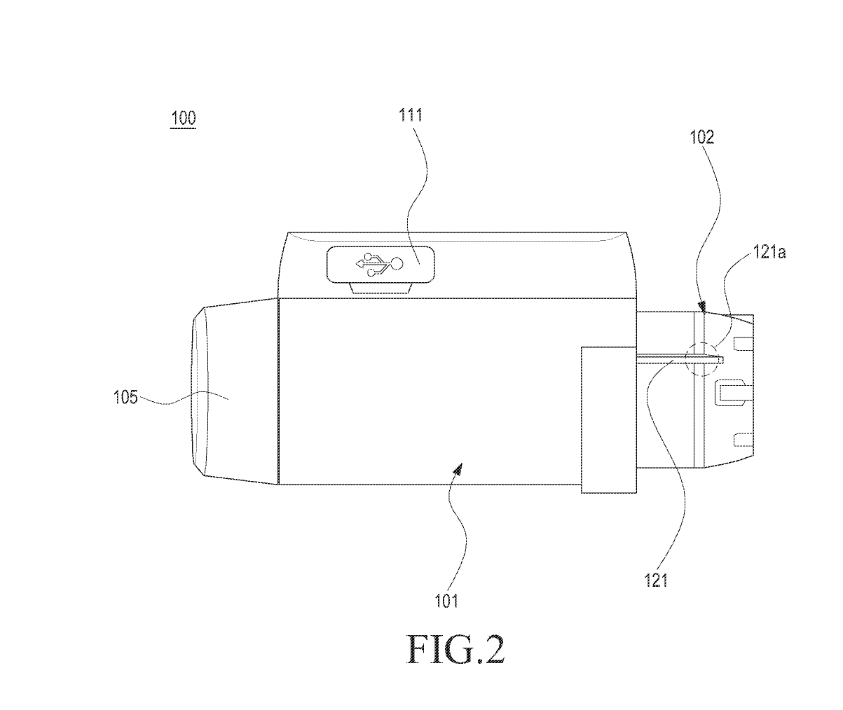

FIG. 1 is a perspective view illustrating a dose measuring device for a drug delivery device according to an embodiment of the present disclosure. FIG. 2 is a side view illustrating a dose measuring device illustrated in FIG. 1 according to an embodiment of the present disclosure.

Referring to FIGS. 1 and 2, a dose measuring device 100 according to an embodiment of the present disclosure may include a housing 101, a rotation ring 103, an encoder, and an auxiliary sleeve 102. The dose measuring device 100 is configured so as to be removable from a drug delivery device (such as the drug delivery device 10 illustrated in FIG. 13). The drug delivery device 10 may be, for example, an insulin pen, and in the following description, the dose measuring device 100 will be described as being removable from the insulin pen.

Although not shown in the drawings, a data storing memory, a communication circuit, a satellite signal receiver, and a processor for controlling the other components of the dose measuring device may be provided in the housing 101. As will be described later, the encoder detects the amount of rotation of the rotation ring 103 and the processor calculates a dose and stores the calculated dose in the memory. In this case, information such as injection date and time, an injection position, and so forth may be received through the communication circuit or the satellite signal receiver and may be stored together with the dose in the memory.

The communication circuit may be manufactured according to a communication standard such as Near Field Communication (NFC), Bluetooth®, or the like. If the communication circuit is configured connectable to a user's mobile communication terminal, the communication circuit may also receive user profile information, such as injection date and time, a dose, temperature/humidity, altitude, air pressure, disease information, and so forth, at the time of injection. If a satellite signal receiver is mounted on the mobile communication terminal, the dose measuring device 100 may receive information about an injection position through the mobile communication terminal even though the satellite signal receiver is not mounted on the housing 101. The user may input information to the memory in advance; this information may include information about a type of a drug, a content of the drug, a manufacturer of the drug, and the like, which are related to a drug delivery device on which the dose measuring device 100 is to be mounted. The information recorded or obtained through the dose measuring device 100, the mobile communication terminal, or wireless communication is recorded and stored by the dose measuring device 100, and states of a patient and a drug are expected from environment information at the time of injection for use as reference information in future medical examination and treatment.

The information recorded at the time of injection may be acquired through various channels. Since the present disclosure is intended to provide a dose measuring device removable from a drug delivery device, obtaining, processing, and storing related information at the time of injection will not be further described.

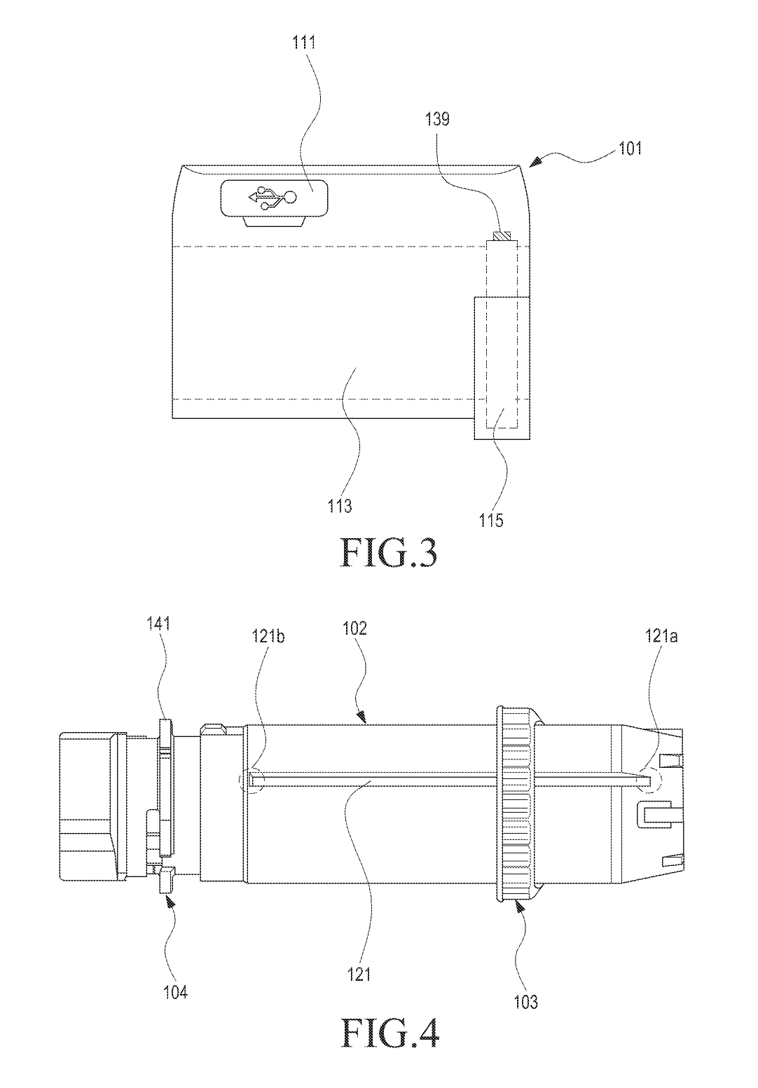

FIG. 3 is a side view illustrating a housing of a dose measuring device according to an embodiment of the present disclosure.

Referring to FIG. 3, the housing 101 may include a through-hole 113 formed to longitudinally pass through the housing 101 from a side end of the housing 101, and the housing 101 receives the rotation ring 103 and the auxiliary sleeve 102 in the through-hole 113. A drug delivery device 10 is longitudinally inserted into the through-hole 113, such that a main body 11 of the drug delivery device 10 is fixed to the housing 101 and a dial sleeve 21 of the drug delivery device 10 is bound to the auxiliary sleeve 102. Although not shown, at least one button and a display device may be provided on a top surface 101a of the housing 101. The button is provided to input information and output stored information at the time of injection, and the progress of execution of a command based on manipulation of the button is output through the display device. The housing 101 may also include a data port 111. The dose measuring device 100 may be connected to a Personal Computer (PC) through a separate cable connected to the data port 111. A user or a doctor may extract information stored in the dose measuring device 100, and may input information regarding a type or a content of a drug to the dose measuring device 100.

A rotation groove 115 is formed in the through-hole 113 of the housing 101 along a circumferential direction of the through-hole 113. The rotation groove 115 is positioned adjacent to the other side end of the housing 101 and receives the rotation ring 103 in such a way to allow rotation of the rotation ring 103. Thus, the rotation ring 103 may rotate in the housing 101, and further on the drug delivery device 10, while being maintained fixed longitudinally.

A rotation detecting element 139 forming a part of the encoder is installed in the housing 101 adjacent to the rotation groove 115. The rotation detecting element 139 may include a 2-way detection switch sensor, an optical sensor, or a Hall-effect sensor to physically contact the rotation ring 103 for detection of the amount of rotation of the rotation ring 103. The structure of the encoder will be further described below.

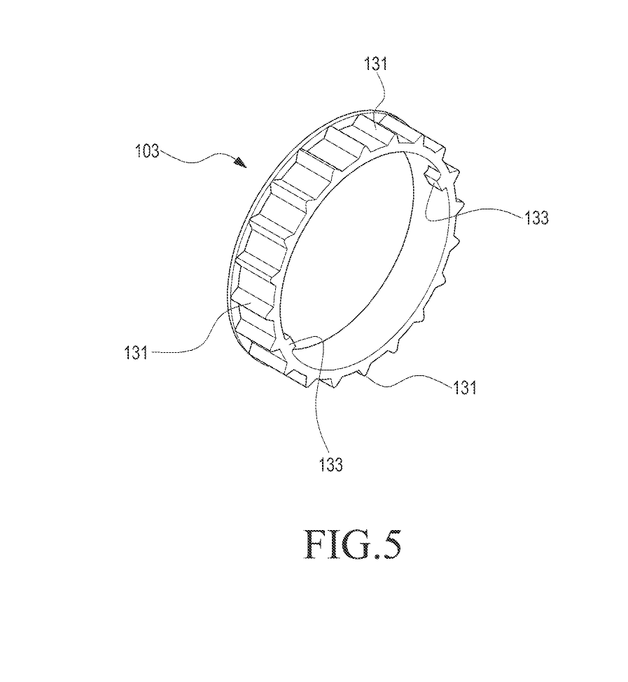

FIG. 4 is a side view illustrating a state in which a housing is removed from a dose measuring device according to an embodiment of the present disclosure. FIG. 5 is a perspective view illustrating a rotation ring of a dose measuring device according to an embodiment of the present disclosure.

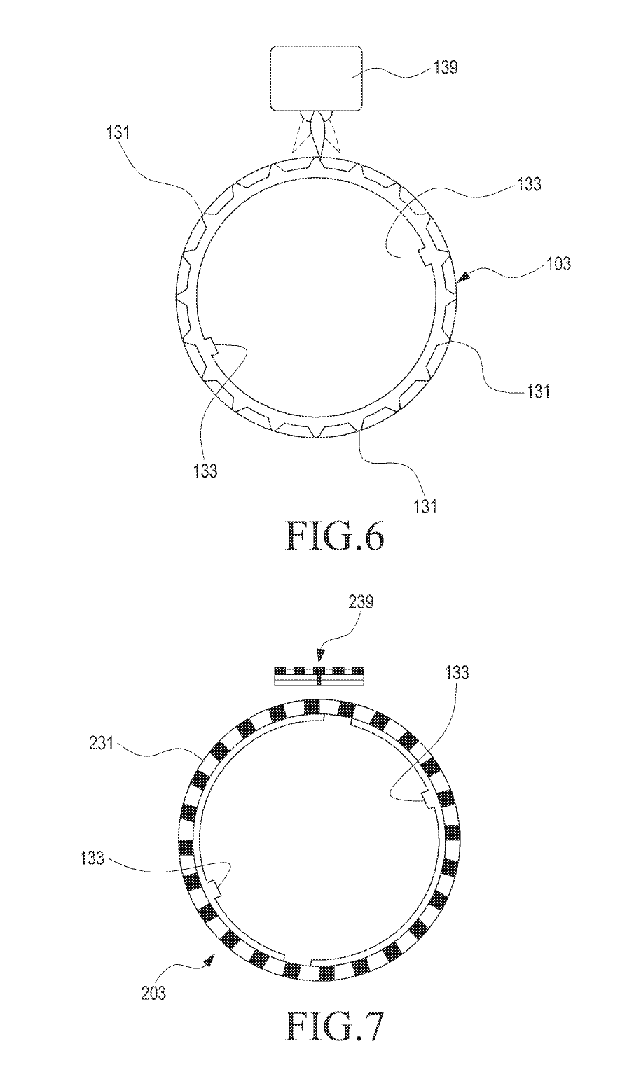

Referring to FIGS. 4 and 5, the rotation ring 103 may include interference protrusions 131 periodically formed on an outer circumferential surface of the rotation ring 103 along a circumferential direction of the rotation ring 103. The rotation ring 103 may also include guide protrusions 133 on an inner circumferential surface of the rotation ring 103. The plurality of guide protrusions 133 may be formed on the inner circumferential surface of the rotation ring 103, and in FIG. 5, a pair of guide protrusions 133 are formed. When the rotation ring 103 is received in the rotation groove 115, the interference protrusions 131 are situated to face an inner wall of the rotation groove 115. The rotation detecting element 139 may be a 2-way detection switch as illustrated in FIG. 6. As the rotation ring 103 rotates, the 2-way detection switch sequentially interferes with the interference protrusions 131, thus repeating on/off operations. As the on/off operations of the 2-way detection switch are performed, the amount of rotation of the rotation ring 103 is detected and the dose measuring device 100 calculates a dose of a drug the user desires to set based on the amount of rotation of the rotation ring 103.

FIGS. 6 and 7 are diagrams for describing an encoder of a dose measuring device according to an embodiment of the present disclosure.

Referring to FIG. 7, the rotation detecting element 139 may be implemented with a non-contact sensor 239, for example, an optical sensor or a Hall-effect sensor, as mentioned above. In this case, a pattern 231 corresponding to the non-contact sensor 239 is formed on the outer circumferential surface of the rotation ring 203. If the non-contact sensor 239 is implemented with an optical sensor, the pattern 231 may include an optical pattern in which a reflector and a non-reflector, for example, a white region and a black region are formed alternately along the circumferential direction of the rotation ring 203. If the non-contact sensor 239 is implemented with a Hall-effect sensor, the pattern 231 may be a magnetic pattern in which the North pole (N) and the South pole (S) are formed alternately along the circumferential direction of the rotation ring 203. The non-contact sensor 239 implemented with the optical sensor or the Hall-effect sensor detects a change in the optical pattern or the magnetic pattern provided on the outer circumferential surface of the rotation ring, and measures the amount and direction of rotation of the rotation ring 203 based on the detected change.

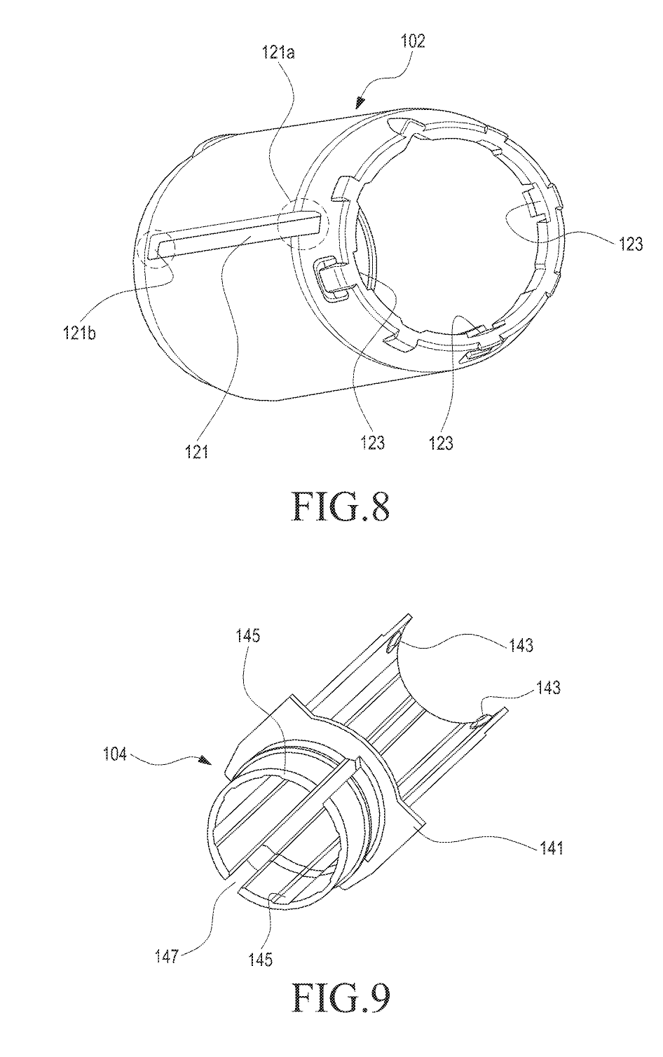

FIG. 8 is a perspective view illustrating an auxiliary sleeve of a dose measuring device according to an embodiment of the present disclosure.

FIG. 8 illustrates the auxiliary sleeve 102. The auxiliary sleeve 102 is received in the housing 101 in such a way to rotate on the housing 101 and linearly move along the longitudinal direction. The auxiliary sleeve 102 is bound to the dial sleeve 21 of the drug delivery device 10. The housing 101 is fixed to the main body 11 of the drug delivery device 10 and the auxiliary sleeve 102 is fixed to the dial sleeve 21 of the drug delivery device 10. The dial sleeve 21 of the drug delivery device 10 linearly moves along the longitudinal direction by spirally moving (i.e., rotating) with respect to the main body 11. Thus, when the dose measuring device 100 is mounted on the drug delivery device 10, the auxiliary sleeve 102 also spirally moves with respect to the housing 101.

Protrusions 25 or grooves may be formed on an outer circumferential surface of the dial sleeve 21 of the drug delivery device 10, or a part of the outer circumferential surface of the dial sleeve 21, such as an adjustment grip 23 is covered with a rubber material having a high friction. The protrusions 25 or the grooves, or the adjustment grip 23 made of a rubber material, are fixed in close contact to an inner side of the auxiliary sleeve 102, such that the auxiliary sleeve 102 is fixed to the dial sleeve 21 of the drug delivery device 10. Binding grooves 123 may be formed on the inner circumferential surface of the auxiliary sleeve 102 to correspond to the shape of the dial sleeve 21. The protrusions 25 formed on the outer circumferential surface of the dial sleeve 21 of the drug delivery device 10 may be used as binding protrusions 25 engaged with the binding grooves 123. However, if the adjustment grip 23 of the dial sleeve 21 is covered with the rubber material and the protrusions 25 are formed on the outer circumferential surface of the adjustment grip 23, the adjustment grip 23 may be fixed in close contact to the inner circumferential surface of the auxiliary sleeve 102 merely with the protrusions 25. Thus, even when the binding grooves 123 are not separately formed on the inner circumferential surface of the auxiliary sleeve 102, the auxiliary sleeve 102 may be bound and fixed to the dial sleeve 21 of the drug delivery device 10.

Guide grooves 121 extending longitudinally are formed on the outer circumferential surface of the auxiliary sleeve 102. The guide grooves 121 receive the guide protrusions 133 of the rotation ring 103. The rotation ring 103 is allowed to rotate in the housing 101, and the auxiliary sleeve 102 rotates with respect to the housing 101, together with the dial sleeve 21 of the drug delivery device 10. As the guide protrusions 133 are engaged with the guide grooves 121, the rotation ring 103 rotates together with the auxiliary sleeve 102. In this way, a linear movement distance of the dial sleeve 21, and further a user-set dose corresponding to adjustment of the dial sleeve 21, may be calculated from the amount of rotation of the rotation ring 103.

The guide groove 121 includes an open end 121a and a closed end 121b. When the auxiliary sleeve 102 moves in a direction of leaving the housing 101, the guide protrusion 133 interferes with the closed end 121b of the guide groove 121. Thus, the auxiliary sleeve 102 cannot leave the housing 101. When the auxiliary sleeve 102 moves in a direction of entering the housing 101 and reaches a limit point, the auxiliary sleeve 102 interferes with a guide member 104 described below and thus cannot move further. The auxiliary sleeve 102 may longitudinally move in a predetermined section on the housing 101.

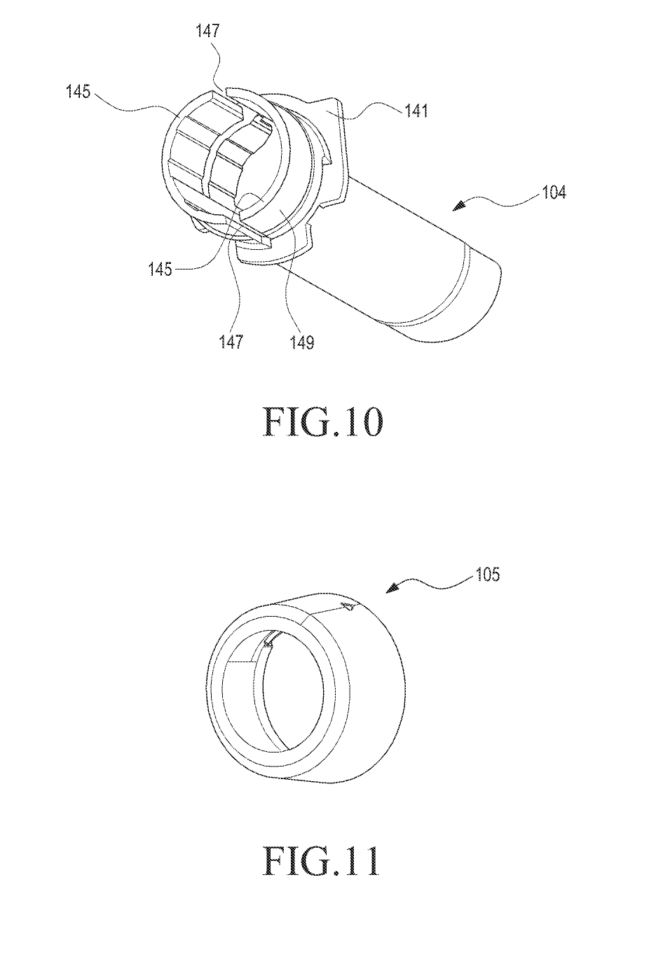

FIGS. 9 and 10 are perspective views illustrating a guide member of a dose measuring device according to an embodiment of the present disclosure.

Referring to FIGS. 9 and 10, the guide member 104 is fixed to the housing 101 to enclose a part of the main body 11 of the drug delivery device 10 and to support and guide rotation and linear movement of the auxiliary sleeve 102. The guide member 104 is generally in a cylindrical shape, but a portion thereof is open. Fixing protrusions 143 are formed on an inner circumferential surface of the guide member 104 and are engaged with the outer circumference surface of the main body 11 of the drug delivery device 10. Grooves corresponding to the fixing protrusions 143 may be formed in the main body 11 of the drug delivery device 10. However, the fixing protrusions 143 may also be engaged with grooves formed for decoration on the main body 11 of the drug delivery device 10.

A dose value printed in the dial sleeve 21 of the drug delivery device 10 may be checked by the user through a display window 15 formed on the main body 11. The display window 15 is configured by opening a portion of the main body 11 or mounting a transparent window on the opened portion. The fixing protrusions 143 may be bound to an edge of the display window 15.

The guide member 104 may include a binding rib 141. The binding rib 141 protrudes from an outer circumferential surface of the guide member 104, and is fixed into the housing 101. The guide member 104 is fixed to the housing 101 and limits a moving range of the auxiliary sleeve 102. Slits 147 are formed in an end of the guide member 104, such that a portion of the guide member 104 includes elastic pieces 145. The elastic pieces 145 may be transformed to a specific degree with respect to the other portions of the guide member 104. When the dose measuring device 100 is mounted on the drug delivery device 10, the elastic pieces 145 closely contact the outer circumferential surface of the drug delivery device 10 to fix the dose measuring device 100 to the drug delivery device 10.

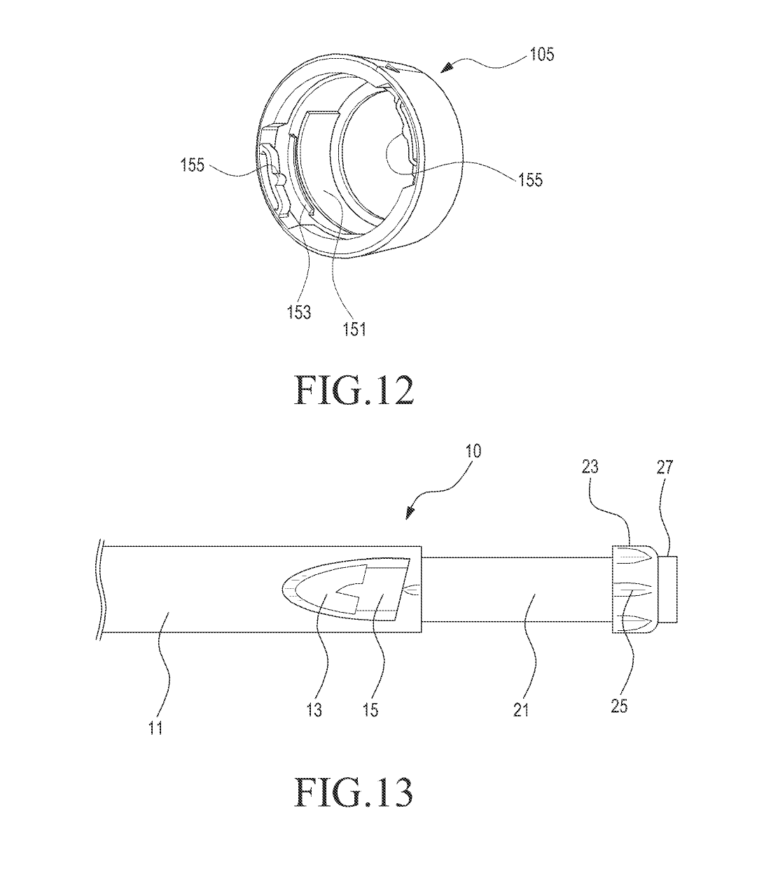

FIGS. 11 and 12 are perspective views illustrating a fixing member of a dose measuring device according to an embodiment of the present disclosure.

Referring to FIGS. 11 and 12, the housing 101 may be completed in such a way that two members are coupled to face each other. To maintain the coupled state between the two members, the dose measuring device 100 may further include a fixing member 105. The fixing member 105 is coupled to an end of the housing 101 in such a way to rotate. Both ends of the fixing member 105 are open and a guide rail 153 is formed on the inner circumferential surface of the fixing member 105. The guide rail 153 is engaged with a groove (not shown) formed on the housing 101 to guide rotation of the fixing member 105 and prevent the fixing member 105 from leaving the housing 101. A first pressurizing protrusion portion 151 and a click protrusion 155 are also provided on the inner circumferential surface of the fixing member 105.

The first pressurizing protrusion portion 151 is formed by a portion of the inner circumferential surface of the fixing member 105 protruding, such that in the portion where the first pressurizing protrusion portion 151 is formed, an inner diameter of the fixing member 105 gently increases or decreases along a rotation direction of the fixing member 105. Once the fixing member 105 is coupled to the housing 101, the first pressurizing protrusion portion 151 is situated to face an outer circumferential surface of an end portion of the guide member 104, more specifically, an outer circumferential surface of the elastic pieces 145.

In this case, as illustrated in FIG. 10, a second pressurizing protrusion portion 149 may be formed in a portion of the outer circumferential surface of the elastic pieces 145. In the portion where the second pressurizing protrusion portion 149 is formed, an outer diameter of the guide member 104 gently increases or decreases along the rotation direction of the fixing member 105. When, by rotating, the fixing member 105 is situated in a position where the first pressurizing protrusion portion 151 and the second pressurizing protrusion portion 149 face each other (hereinafter, a ‘lock position’), the elastic pieces 145 move into the guide member 104. If the dose measuring device 100 is mounted on the drug delivery device 10, the elastic pieces 145 in the lock position move into the guide member 104, thus being fixed in close contact to the outer circumferential surface of the main body 11 of the drug delivery device 10. As the fixing member 105 rotates, the guide member 104, and further the dose measuring device 100 may be completely fixed to the drug delivery device 10.

Click protrusions 155 are elastically supported on the inner circumferential surface of the fixing member 105. As the fixing member 105 rotates, the click protrusions 155 slide and contact the outer circumferential surface of the housing 101. Although not shown, click grooves (not shown) engaged with the click protrusions 155 may be formed along moving tracks of the click protrusions 155 on the outer circumferential surface of the housing 101. In the lock position, the click protrusions 155 may be engaged with one of the click grooves.

As mentioned above, the thicknesses of the first pressurizing protrusion portion 151 and the second pressurizing protrusion portion 149 gradually increase or decrease along the rotation direction of the fixing member 105. With the elastic pieces 145 and the first and second pressurizing protrusion portions 151 and 149, the fixing member 105 has a tendency to rotate in a direction of leaving the lock position. Such a rotation tendency of the fixing member 105 may be amplified when the dose measuring device 100 is mounted on the drug delivery device 10. Thus, if the click protrusions 155 are engaged with the clock groove formed on the housing 101, rotation of the fixing member 105 may be suppressed. If the fixing member 105 rotates to leave the lock position by means of the first and second pressurizing protrusion portions 151 and 149 and the elastic pieces 145, then the dose measuring device 100 may not be stably fixed to the drug delivery device 10. Moreover, when the drug delivery device 10 or a drug cartridge is replaced, the user may release the lock state by merely rotating the fixing member 105 and thus may easily remove the dose measuring device 100 from the drug delivery device 10.

FIG. 13 illustrates a drug delivery device configured removable from a dose measuring device according to an embodiment of the present disclosure. FIG. 13 illustrates the drug delivery device 10 on which the dose measuring device 100 is mounted.

Referring to FIG. 13, the drug delivery device 10 may include the main body 11 longitudinally extending in a cylindrical shape and the dial sleeve 21 coupled to the main body 11 to rotate. As a portion of the main body 11 is opened to form the display window 15, and a groove 13 is formed around the display window 15 for decoration. As described previously, the fixing protrusions 143 formed on the guide member 104 may configured to be engaged with the edge of the display window 15 or the groove 13. The guide member 104 may be provided to enclose a portion of the main body 11, while exposing at least the display window 15. By doing so, when adjusting a dose, the user may see the dose corresponding to rotation of the dial sleeve 21 through the display window 15.

The adjustment grip 23 is provided in an end portion of the dial sleeve 21, and the binding protrusions 25 are formed on the outer circumferential surface of the adjustment grip 23. As mentioned above, the outer circumferential surface of the adjustment grip 23 may be covered with a rubber material, and the outer circumferential surface of the adjustment grip 23 is substantially fixed in close contact to the inner circumferential surface of the auxiliary sleeve 102. An injection button 27 may also be provided in an end portion of the dial sleeve 21. The user rotates the dial sleeve 21 by using the adjustment grip 23 to adjust and select a dose of a drug, rotates the dial sleeve 21 to correspond to the selected dose, and then presses the injection button 27 to operate the drug delivery device 10.

The auxiliary sleeve 102 may be manufactured with a transparent material. As stated above, to adjust a dose, the user sees the dose through the display window 15, and in this state, the auxiliary sleeve 102 is coupled to the housing 101 while enclosing the main body 11. In this way, since the auxiliary sleeve 102 is manufactured with the transparent material, the user may see the adjusted dose through the display window 15. However, the dose may be calculated based on the amount of rotation of the rotation ring 103, such that the adjusted dose may be displayed on the display device of the dose measuring device 100. Accordingly, in some products, the display window 15 may be hidden when the dose measuring device 100 is coupled to the drug delivery device 10.

The drug delivery device 10 is longitudinally inserted from a side of the housing 101 into the housing 101, more specifically, the through-hole 113. Once the drug delivery device 10 is entirely inserted into the housing 101, the adjustment grip 23 of the dial sleeve 21 is partially bound to the inner circumferential surface of the auxiliary sleeve 102 and partially protrudes from an end portion of the auxiliary sleeve 102. Thus, the injection button 27 is exposed on the end portion of the auxiliary sleeve 102. In this case, the binding protrusions 25 may be fixed in close contact to the inner side of the auxiliary sleeve 102 or may be fixed engaged to the binding grooves 123. As a result, the auxiliary sleeve 102 rotates, together with the dial sleeve 21, with respect to the main body 11.

When the drug delivery device 10 is entirely inserted into the housing 101, the fixing protrusions 143 are engaged with the outer circumferential surface of the main body 11, for example, the edge of the display window 15, such that the guide member 104 is fixed to the main body 11. In addition, once the fixing member 105 rotates to the lock position, the elastic pieces 145 pressurize the outer circumferential surface of the main body 11 and are fixed in close contact to the outer circumferential surface of the main body 11. If the pair of elastic pieces 145 are formed to face each other, the guide member 104 may be more firmly fixed to the main body 11. In the lock position, the click protrusions 155 of the fixing member 105 are engaged with the clock groove formed in the housing 101, such that the fixing member 105 stably maintains the state of being fixed to the housing 101. If the user rotates the fixing member 105 forcibly, the click protrusions 155 leave the click groove.

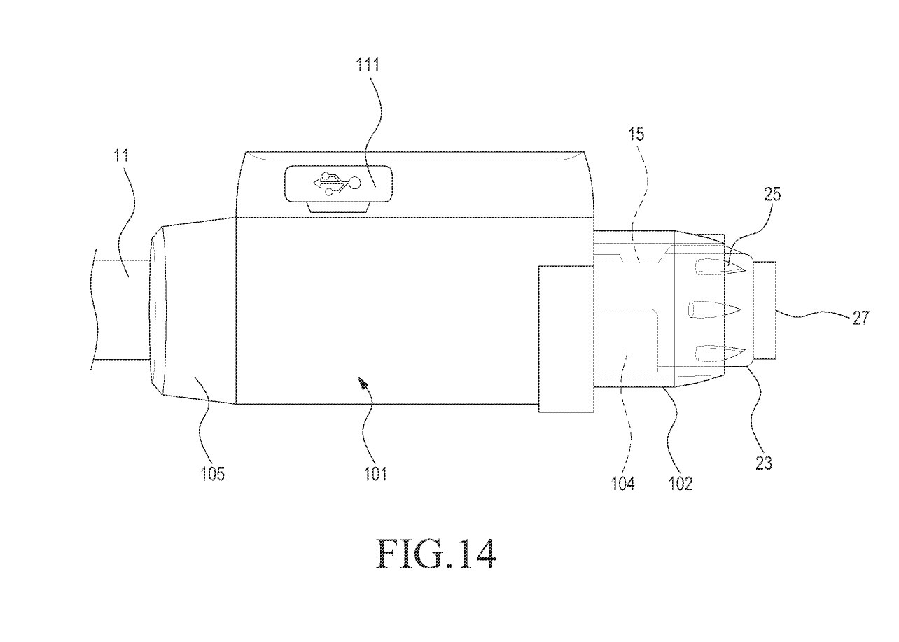

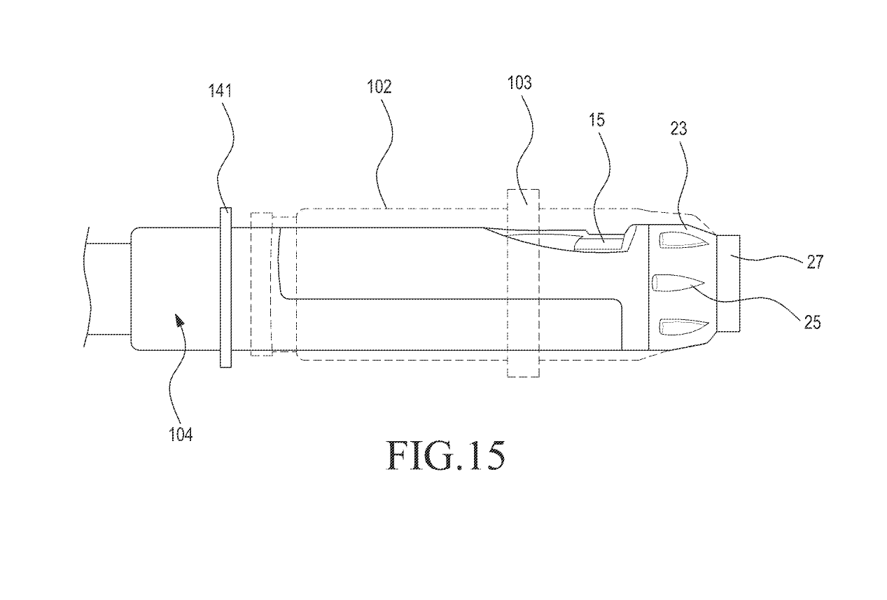

FIG. 14 is a side view illustrating a state in which a dose measuring device is mounted on a drug delivery device according to an embodiment of the present disclosure. FIG. 15 is a side view illustrating a state in which a housing is removed from a dose measuring device according to an embodiment of the present disclosure. FIGS. 14 and 15 illustrate a state in which the dial sleeve 21 entirely enters the main body 11, and FIG. 13 illustrates a state where the dial sleeve 21 is withdrawn from the main body 11 by rotating.

Referring to FIGS. 14 and 15, if the user rotates the adjustment grip 23 or the auxiliary sleeve 102 while the dial sleeve 21 completely enters the main body 11, the dial sleeve 21 is gradually withdrawn from the main body 11 by rotating. As the dial sleeve 21 rotates, the auxiliary sleeve 102 also rotates together. Accordingly, the auxiliary sleeve 102 is also gradually withdrawn from the housing 101.

As stated above, the rotation ring 103 may rotate together with the auxiliary sleeve 102 by means of the guide protrusions 133 and the guide grooves 121, and is fixed onto the housing 101 in the longitudinal direction. Accordingly, the rotation ring 103 rotates in the housing 10 as the auxiliary sleeve 102 rotates. The encoder, which is implemented with a combination of arrangement of the interference protrusions 131, the optical pattern or the magnetic pattern provided on the outer circumferential surface of the rotation ring 103, and the rotation detecting element 139 or the non-contact sensor 239 corresponding to the pattern, detects the amount of rotation of the rotation ring 103. Thus, the linear movement distance of the auxiliary sleeve 102 and the dial sleeve 21, as well as the adjusted dose may be calculated.

To remove the dose measuring device 100 from the drug delivery device 10, the user rotates the fixing member 105 to cause the fixing member 105 to leave the lock position. Once the fixing member 105 leaves the lock position, the user moves the drug delivery device 10 in the direction of being inserted into the housing 101, thus easily separating the drug delivery device 10 and the dose measuring device 100 from each other.

The dose measuring device structured as described above may be mounted on and removed from a drug delivery device that has been already commercialized but has no dose measuring and storing functions, for example, an insulin pen. Therefore, the user may measure or acquire information such as a dose by using the dose measuring device according to the present disclosure and automatically store the information. Accordingly, the user or a doctor of the user may determine a change in the symptoms of the user by referring to information recorded and stored in the dose measuring device according to the present disclosure.

Other effects that may be obtained or expected from the embodiment of the present disclosure are explicitly or implicitly disclosed in the detailed description of the embodiment of the present disclosure. Various effects expected from the embodiment of the present disclosure have been disclosed in the detailed description of the present disclosure.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A dose measuring device for a drug delivery device that comprises a main body and a dial sleeve coupled to the main body to spirally move, the dose measuring device comprising:
a housing arranged to receive the main body of the drug delivery device;
a rotation ring arranged in the housing to rotate;
an encoder installed in the housing to detect an amount of rotation of the rotation ring; and
an auxiliary sleeve arranged to be removable from the dial sleeve of the drug delivery device and to spirally move together with the dial sleeve in the housing, wherein the rotation ring is bound to the auxiliary sleeve to rotate together with the auxiliary sleeve and is fixed in the housing in a longitudinal direction of the main body.

2. The dose measuring device of claim 1, wherein the drug delivery device is inserted into the housing from a side of the housing in the longitudinal direction of the main body, such that an end portion of the dial sleeve is bound to an inner side of the auxiliary sleeve and the housing is bound onto the main body.

3. The dose measuring device of claim 1,
wherein the auxiliary sleeve spirally moves together with the dial sleeve while binding protrusions formed on an outer circumferential surface of the dial sleeve are engaged to an inner surface of the auxiliary sleeve.

4. The dose measuring device of claim 2,
wherein the auxiliary sleeve spirally moves together with the dial sleeve while binding protrusions formed on an outer circumferential surface of the dial sleeve are engaged to an inner surface of the auxiliary sleeve.

5. The dose measuring device of claim 1, wherein the encoder comprises:
interference protrusions periodically formed on an outer circumferential surface of the rotation ring in a circumferential direction of the rotation ring; and
a 2-way detection switch installed on the housing,
wherein the 2-way detection switch repeats on/off operations as the 2-way detection switch interferes with the interference protrusions along with the rotation of the rotation ring.

6. The dose measuring device of claim 1, wherein the encoder comprises:
a magnetic pattern periodically formed on an outer circumferential surface of the rotation ring in a circumferential direction of the rotation ring; and
a Hall-effect sensor installed on the housing,
wherein the Hall-effect sensor detects a change in the magnetic pattern as the rotation ring rotates.

7. The dose measuring device of claim 1, wherein the encoder comprises:
an optical pattern periodically formed on an outer circumferential surface of the rotation ring in a circumferential direction of the rotation ring; and
an optical sensor installed on the housing,
wherein the optical sensor detects a change in the optical pattern as the rotation ring rotates.

8. The dose measuring device of claim 1, further comprising:
at least one guide groove extending longitudinally on an outer circumferential surface of the auxiliary sleeve; and
a guide protrusion formed on an inner circumferential surface of the rotation ring to be engaged with the guide groove,
wherein as the guide protrusion is engaged with the guide groove, the rotation ring rotates together with the auxiliary sleeve and is fixed in the housing in the longitudinal direction of the main body.

9. The dose measuring device of claim 8, wherein an end of the guide groove is an open end and the other end of the guide groove is a closed end.

10. The dose measuring device of claim 1, further comprising:
a guide member fixed to an inner circumferential surface of the housing such that at least a portion of the main body is received in the guide member,
wherein the guide member extends in the longitudinal direction of the main body to guide spiral movement of the auxiliary sleeve.

11. The dose measuring device of claim 10, wherein the guide member comprises an elastic piece that closely contacts the outer circumferential surface of the main body.

12. The dose measuring device of claim 10, further comprising:
a fixing member rotatably coupled to an end of the housing; and
an elastic piece formed in an end portion of the guide member and covered with the fixing member,
wherein the fixing member pressurizes the elastic piece to cause the elastic piece to closely contact an outer circumferential surface of the main body.

13. The dose measuring device of claim 12, further comprising:
a first pressurizing protrusion portion formed on an inner circumferential surface of the fixing member; and
a second pressurizing protrusion portion formed on an outer circumferential surface of the elastic piece,
wherein as the fixing member rotates, the elastic piece closely contacts the outer circumferential surface of the main body in a position where the first pressurizing protrusion portion faces the second pressurizing protrusion portion.

14. The dose measuring device of claim 10, further comprising:
a fixing protrusion formed on an inner circumferential surface of the guide member,
wherein the guide member is fixed to the main body as the fixing protrusion is engaged on the outer circumferential surface of the main body.

15. The dose measuring device of claim 1, further comprising:
a rotation groove circumferentially formed on the inner circumferential surface of the housing to rotatably receive the rotation ring.

16. The dose measuring device of claim 1, wherein the dose measuring device records and stores information regarding a patient's profile, the profile including injection date and time, a type and a dose of an injected drug, an injection position and a place where injection is performed, temperature and humidity at the time of injection, altitude and air pressure at the time of injection, and disease information at the time of injection.

17. The dose measuring device of claim 16, further comprising:
a memory configured to store the profile;
a communication unit; and
a processor configured to receive information for inclusion in the profile from an external source via the communication unit and to store the profile in the memory.

18. The dose measuring device of claim 17, further comprising:
an input unit;
wherein the processor stores information input via the input unit in the profile stored in the memory.

19. A drug delivery device, comprising:
a main body;
a dial sleeve coupled to the main body so as to rotate and including an adjustment grip on one end portion; and
an injection button pressable by the user to operate the drug delivery device to deliver a drug to the user's body,
wherein binding protrusions are formed on an outer circumferential surface of the dial sleeve so as to engage with an inner surface of an auxiliary sleeve of a dose measuring device, such that the auxiliary sleeve of the dose measuring device spirally moves together with the dial sleeve.

20. The drug delivery device of claim 19, wherein the main body further comprises grooves arranged on an outer circumferential surface of the main body such that at least a portion of the main body is receivable within a housing of the dose measuring device so as to guide spiral movement of the auxiliary sleeve.
